(19) United States
(12) Patent Application Publication
(10) Pub. No.: US 2025/0302631 A1
(43) Pub. Date: Oct. 2, 2025

(54) HUMERAL ANCHOR COMPONENT OF SHOULDER PROSTHESIS

(71) Applicant: FX SHOULDER SOLUTIONS, VIRIAT (FR)

(72) Inventors: Maxime ANTONI, Brunstatt (FR); Giorgio GRESTA, SAINT JUST-SAINT RAMBERT (FR); Howard HARRIS, Southlake, TX (US); Geoffroy NOURISSAT, Vincennes (FR); Umasuthan SRIKUMARAN, Ellicott City, MD (US)

(73) Assignee: FX SHOULDER SOLUTIONS, VIRIAT (FR)

(21) Appl. No.: 19/092,594

(22) Filed: Mar. 27, 2025

(30) Foreign Application Priority Data
Mar. 27, 2024 (FR) 2403065

(51) Int. Cl. A61F 2/30; A61F 2/40

(52) U.S. Cl. CPC A61F 2/30749; A61F 2002/30649; A61F 2002/4062

(57) ABSTRACT

The present disclosure relates to a humeral anchor component of a humeral joint part of a shoulder prosthesis. The humeral anchor component includes a main body intended to be inserted inside a humerus from a resected metaphyseal surface, and a proximal flange forming a proximal end of the humeral anchor component and placed at one end of the main body, a proximal face of the proximal flange extending in the humerus and underneath the resected metaphyseal surface.

### TECHNICAL FIELD

The present disclosure relates generally to a humeral anchor component for anchoring to a humerus of a humeral joint part of a shoulder prosthesis.

### BACKGROUND

It is known to anchor a humeral joint part using a tapered medullary rod which include the humeral joint part. The medullary rod has a flared proximal section, intended to be positioned at the bone metaphysis, and a tapered distal section intended to be inserted in the medullary canal up to the diaphysis. Such a joint part with medullary rod may however be difficult to implant when the bone quality is not good, particularly in case of osteoporosis on an elderly patient. The same is true when replacing a prosthesis implanted previously, this previous implantation having already resulted in a more or less extensive bone ablation. In both cases, attachment of the joint part to the bone may not be perfect and may not perfectly withstand the repeated stresses to which the prosthesis is subjected over time.

In another situation, and for a patient having a healthy bone (for example, a younger patient) but a joint worn by numerous stresses (for example due to work or sport) and/or for whom an operation of the rotator cuff muscles is not or no longer possible, improving the resistance over time of the prostheses allows the installation of a prosthesis by using an anchor component without a medullary rod. This is due to the fact that the prosthesis can be stabilized without having to find an anchor point that is low enough in the diaphyseal section of the humerus since the quality of the epiphysis and the metaphysis is good. The anchor component then extends in the metaphyseal section of the bone, after resection of the humeral head.

In the case of a reverse prosthesis, the stack of the components forming the humeral joint part may result in a displacement of the center of rotation of the humerus joint towards the glenoid. This is not optimum since, without a prosthesis, the center of rotation of the anatomical joint is located at the humerus, not at the glenoid. Installing a prosthesis may therefore result in a displacement of the center of rotation of the joint to create a situation which does not reproduce the anatomical joint situation.

### SUMMARY

The main objective of the disclosed embodiments is to overcome this disadvantage by providing a humeral anchor component in order to keep the position of the center of rotation of the joint as close as possible to an anatomical reality of the joint while implementing an anchor component that is as least invasive as possible.

Thus, the present disclosure relates to a humeral anchor component of a humeral joint part of a shoulder prosthesis, the humeral anchor component comprising:
a main body intended to be inserted inside a humerus from a resected metaphyseal surface, and
a proximal flange forming a proximal end of the humeral anchor component and placed at one end of the main body, a proximal face of the proximal flange extending in the humerus and underneath the resected metaphyseal surface.

Thus, the anchor component is placed fully inside the humerus and underneath the resected metaphyseal surface. The entire humeral joint part can therefore be moved towards the humerus in case of a reverse shoulder prosthesis (anchor component intended to receive a cup). This displacement of the joint towards the humerus allows, when this is not the case for a prosthesis according to the prior art, a displacement of the center of rotation of the joint towards the humerus in order to reproduce an anatomical situation in which the center of rotation of the joint is located at the humerus, not at the glenoid. Whatever the prosthesis (anatomical or reverse), an anchor component that is as least invasive as possible is obtained.

According to other optional characteristics of the humeral anchor component, taken alone or in combination:
The proximal flange is intended to extend substantially parallel to a plane in which the resected metaphyseal surface extends,
the proximal face of the proximal flange is intended to extend in the humerus at a distance of between 1 and 10 millimeters underneath the resected metaphyseal surface, preferably between 1 and 5 millimeters underneath the resected metaphyseal surface,
the main body is intended to extend only in a metaphyseal section of the humerus, the main body comprising a peripheral retention system formed by several peripheral anchor plates separated from each other and intended to extend from the proximal flange towards a distal section of the humerus, two adjacent peripheral anchor plates extending in planes that are not parallel to each other,
the peripheral anchor plates extend in a direction substantially perpendicular to a plane in which the proximal face of the proximal flange extends,
the width of the peripheral anchor plates is irregular along their extension direction,
the main body comprises at least one stabilization stud intended to extend from the proximal flange towards a distal section of the humerus,
the stabilization stud extends in a direction substantially perpendicular to a plane in which the proximal face of the proximal flange extends,
the stabilization stud comprises retention grooves extending substantially parallel to a longitudinal axis of the stabilization stud,
the stabilization stud is a hollow body open at its distal end and comprises a plurality of through-holes formed in its wall,
the stabilization stud is a hollow tubular body open at its proximal end and comprises a tapping formed on an internal surface of the stabilization stud, and
the stabilization stud comprises at least one lateral ring extending from an external face of the stabilization stud in a direction substantially perpendicular to a longitudinal axis of the stabilization stud.

The present disclosure also relates to a humeral joint part of a shoulder prosthesis comprising a humeral anchor component according to an embodiment.

The humeral joint part may include a replacement humeral head, and advantageously a spacer placed between the humeral anchor component and the replacement humeral head.

Alternatively, the humeral joint part may include a cup for receiving a glenosphere.

The humeral joint part may include a diaphyseal section, the diaphyseal section typically being formed by a humeral rod.

The humeral joint part may further include a male Morse cone attached to the diaphyseal section and a female Morse cone carried by the humeral anchor component.

The present disclosure also relates to a kit for implanting a humeral joint part according to an embodiment, the kit comprising several humeral anchor components according to the embodiment of different diameters and several diaphyseal sections of different lengths.

The implantation kit may include at least one screw for attaching a humeral anchor component to a diaphyseal section.

The implantation kit may include at least one male Morse cone intended to be attached to the diaphyseal section.

Advantageously, the implantation kit may include at least one spacer and at least one replacement humeral head and/or at least one cup.

The present disclosure also relates to a shoulder prosthesis comprising a humeral joint part according to an embodiment.

The shoulder prosthesis can be an anatomical prosthesis or a reverse prosthesis.

### BRIEF DESCRIPTION OF THE FIGURES

The various disclosed embodiments will be better understood on reading the following description, given solely by way of example and with reference to the accompanying drawings in which:

FIG. 1 is a first perspective view of a humeral anchor component according to an embodiment,

FIG. 2 is a second perspective view of a humeral anchor component according to an embodiment,

FIG. 3 is a side view of a humeral anchor component according to an embodiment,

FIG. 4 is a longitudinal cross-sectional view of a humeral anchor component according to an embodiment,

FIG. 5 is an exploded and perspective view of a humeral joint part according to a first variant of the first embodiment,

FIG. 6 is an assembled and side view of the humeral joint part according to the first variant of the first embodiment,

FIG. 7 is an exploded and perspective view of a humeral joint part according to a second variant of the first embodiment,

FIG. 8 is an exploded and perspective view of a reverse shoulder prosthesis comprising a humeral joint part according to the second variant of the first embodiment,

FIG. 9 is an assembled and side view of a reverse shoulder prosthesis comprising a humeral joint part according to the second variant of the first embodiment,

FIG. 10 is a side view of a humeral joint part according to the second variant of the first embodiment that is implanted in a humerus,

FIG. 11 is a side view of a set of humeral joint parts according to a second embodiment, and

FIG. 12 is a global algorithm of a prosthesis according to the second embodiment, the prosthesis being an anatomical or reverse prosthesis.

### DETAILED DESCRIPTION

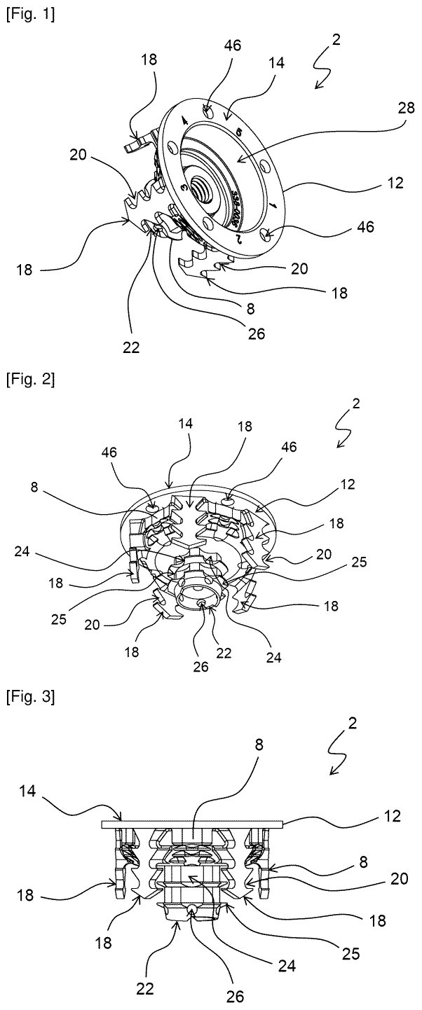

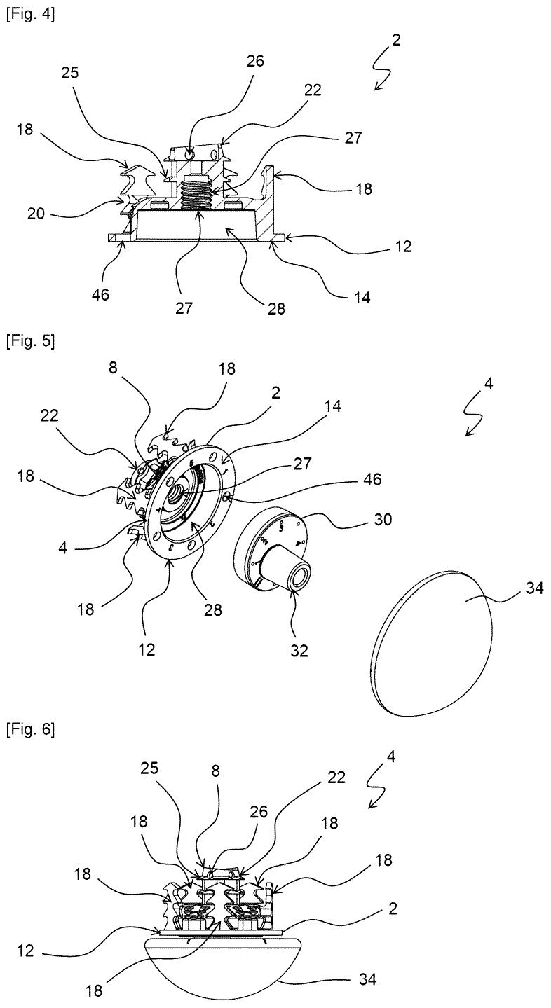

FIGS. 1 to 4 show a humeral anchor component 2 of a humeral joint part 4 or 4′ (see FIGS. 5 to 9) of a shoulder prosthesis (see FIGS. 8 and 9: exploded and assembled views of a reverse shoulder prosthesis). A humeral anchor component means a section of a humeral joint part intended to be implanted in a cavity formed in a proximal end of a humerus, whether the humeral joint part is that of an anatomical or reverse shoulder prosthesis. The humeral anchor component 2 can be made from a biocompatible metal such as a titanium or stainless steel alloy.

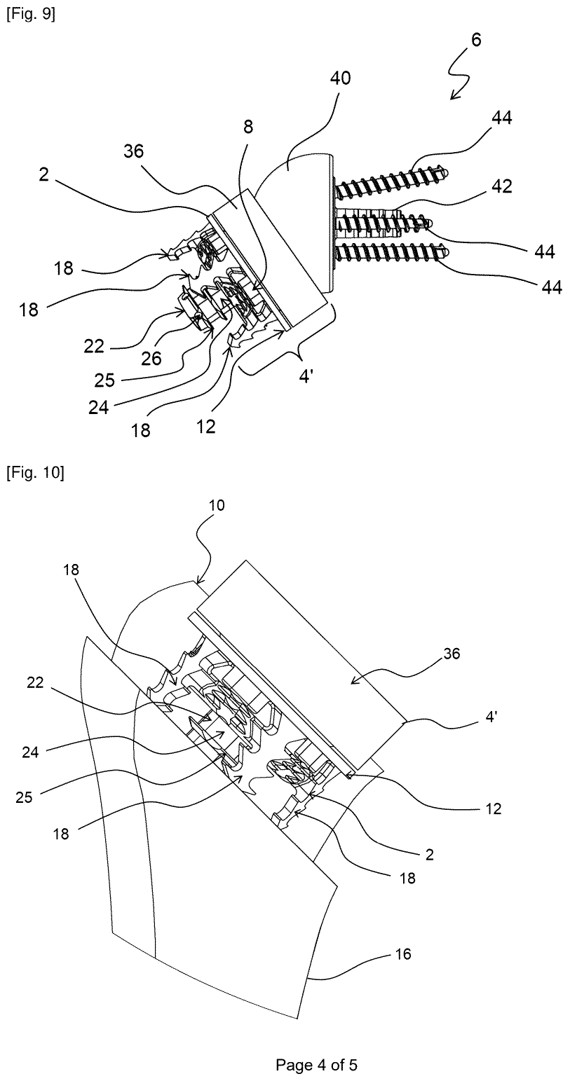

The humeral anchor component 2 includes a main body 8 intended to be inserted inside the humerus from a resected metaphyseal surface 10 (see FIG. 10). It also comprises a proximal flange 12 placed at a proximal end of the main body 8 and that can be intended to extend substantially parallel to the resected metaphyseal surface 10.

A proximal face 14 of the proximal flange 12 extends in the humerus and underneath the resected metaphyseal surface 10. This means that the proximal end of the humeral anchor component 2, intended to be placed as close as possible to the proximal end of the humerus, is located inside the humerus and under the plane formed by the resected metaphyseal surface 10. As explained above, this is carried out to “bury” the humeral anchor component 2 in the humerus and therefore to move the humeral joint part and consequently the shoulder prosthesis towards the humerus, in order to place the center of rotation of the joint as close as possible to the humerus.

The proximal face 14 of the proximal flange 12 can for example extend in the humerus at a distance of between 1 and 10 millimeters underneath the resected metaphyseal surface 10, preferably between 1 and 5 millimeters underneath the resected metaphyseal surface.

The diameter of the proximal flange 12 is between 30 and 48 millimeters.

Advantageously, and according to a first embodiment, the main body 8 extends only in a metaphyseal section of the humerus 16. This means that the anchor component 2 does not have an anchor medullary rod. This is particularly advantageous, as explained above, for patients having a healthy metaphyseal section of the humerus that can be used as anchor base for the humeral anchor component 2 and that can in particular be placed against the calcar of the humeral head which is advantageously retained during the bone resection. Another way of implanting an anchor component according to the first embodiment corresponds to an implantation for a patient whose bone quality is not optimum, but having a deformation of the humerus preventing the installation of a diaphyseal section, typically of a humeral rod.

The main body 8 may include a peripheral anchor system including several peripheral anchor plates 18 separated from each other, five in the example shown on the figures, intended to extend from the proximal flange 12 to a distal section of the humerus 16. In the example shown, the latter extend from the proximal flange 12 substantially perpendicular to a plane in which the proximal face 14 of the proximal flange 12 extends, two adjacent peripheral anchor plates 18 extending in planes that are not parallel to each other. The advantage of using a peripheral anchor is to create said anchor in the cortical section of the humerus which is the densest part of the bone. Thus, the anchoring of the humeral anchor component 2 in the humerus 16 is optimized by interaction with the hardest part of the humerus 16. Installing peripheral anchor plates 18 with adjacent plates not extending in planes parallel to each other prevents the anchor component 2 from rotating in the humerus 16.

Advantageously, the width of the peripheral anchor plates 18 can be irregular along their extension direction. This can be seen on the figures on which indentations 20 are present on each side of the peripheral extension plates 18. These irregularities ensure that the humeral anchor component 2 is securely anchored in the bone.

Preferably, one or more portions of the sides of the peripheral anchor plates extend in a direction parallel to an extension direction of the proximal face 14 of the proximal flange 12. This can be seen on the figures on which each side of each peripheral anchor plate 18 comprises three portions extending in such a direction. Once again, this optimizes the anchoring of the anchor component 2 in the humerus 16.

The length of the peripheral anchor plates 18 is advantageously between 12 and 20 millimeters.

The main body 8 can comprises a stabilization stud 22 placed centrally in the example of the figures, intended to extend from the proximal flange 12 towards a distal section of the humerus 16. In the example shown, the latter extends from the proximal flange 12 substantially perpendicular to a plane in which the proximal face 14 of the proximal flange 12 extends. This stabilization stud 22 prevents inversion of the anchor component 2 and therefore of the joint part 4 or 4′ in the humerus 16.

The length of the stabilization stud is advantageously between 14 and 30 millimeters.

The stabilization stud 22 can advantageously comprise retention grooves 24 extending substantially parallel to a longitudinal axis of the stabilization stud 22. The latter optimize the anti-inversion function of the stabilization stud 22.

The stabilization stud 22 can also be a hollow body open at its distal end (potentially at its proximal end and at its distal end) and comprise through-holes 26 formed in its wall. The latter connect the inside and the outside of the stabilization stud 22. This allows bone colonization and vascularization of the bone portion located inside the stabilization stud 22. This vascularization ensures that a dense bone is preserved inside the stabilization stud 22, which optimizes stabilization of the humeral anchor component 2 in the humerus 16 by allowing anchoring from the inside of the stabilization stud 22.

The stabilization stud 22 can also be a hollow body open at its proximal end (potentially at its proximal end and at its distal end) and comprise a tapping 27 formed on an internal surface of the stabilization stud 22. This tapping 27 allows the humeral anchor component 2 to be removed from the humerus 16 if this should be necessary, by screwing a tool to extract the humeral anchor component 2. An attachment system other than a tapping could be used, for example a system with teeth or notches.

The stabilization stud can also comprise at least one lateral ring 25 (two on the figures) extending from an external face of the stabilization stud 22 in a direction substantially perpendicular to a longitudinal axis of the stabilization stud 22. The lateral ring also ensures that the humeral anchor component 2 is securely anchored in the bone. The lateral ring 25 can also comprise notches aligned with the retention grooves 24 in order to preserve their function. The retention grooves are generally not continuous so that bone is positioned between the portions of the retention grooves 24 to further prevent the rotation of the humeral anchor component 2.

The humeral anchor component 2 can also comprise a recess 28 that can form a conical attachment system intended to receive another portion of a humeral joint part 4 or 4′ (cup or spacer) as will be described below.

The proximal flange 12 can also comprise stitching holes 46 (shown on FIGS. 1 to 4) that can be used to attach the soft parts of the bone to the humeral anchor component 2.

FIG. 5 shows a humeral joint part 4 according to a first embodiment and in the case where the shoulder prosthesis is an anatomical prosthesis, in other words reproducing the shape of the anatomical joint. The humeral joint part 4 comprises, in addition to the humeral anchor component 2, a spacer 30 intended to be placed in the recess 28. The spacer 30 comprises an attachment stud 32 intended to receive a replacement humeral head 34.

FIG. 6 shows a humeral joint part 4 according to a first variant of the first embodiment and in the case where the shoulder prosthesis is an anatomical prosthesis, the humeral joint part 4 being assembled. The spacer 30 is placed in the recess 28 and the reception stud 32 is inserted in an attachment hole formed in the replacement humeral head 34.

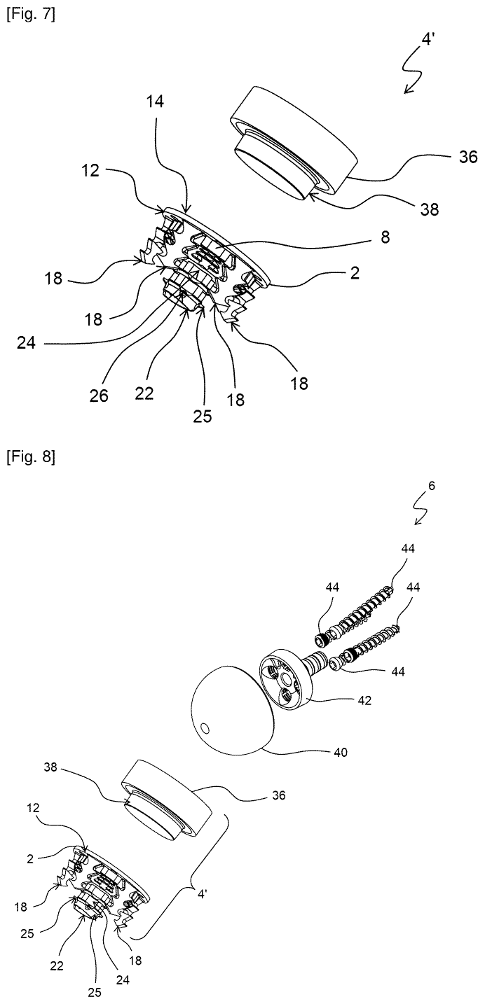

FIG. 7 shows a humeral joint part 4′ according to a second variant of the first embodiment. In this embodiment, the humeral joint part 4′ is that of a reverse shoulder prosthesis 6 shown on FIG. 8. A cup 36 is intended to be installed on the humeral anchor component 2. This cup 36 comprises, at its end placed opposite the glenoid, a concave surface intended to receive a glenosphere 40 (shown on FIGS. 8 and 9). Cups of different height can be used with the same humeral anchor component 2.

In this embodiment, a base 38 of the cup 36 is intended to be placed in the recess 28. The base 38 can be conical and quite wide, with for example a diameter at the gauge plane of between 15 and 30 millimeters.

The cup 36 is advantageously as wide as possible in order to form a concave surface that is as deep as possible. This makes it possible to have a layer of polyethylene in the concave surface, 4 millimeters thick for example, against which the glenosphere 40 is placed while allowing the glenosphere 40 to be placed as deeply as possible in the concave surface.

FIGS. 8 and 9 show a reverse shoulder prosthesis 6, respectively an exploded view (FIG. 8) and an assembled view (FIG. 9). It comprises the humeral joint part 4′, and a glenoid joint part comprising in the example shown the glenosphere 40, a metaglene 42 intended to be attached in the glenoid and on which the glenosphere 40 is attached. Several anchor screws 44 are intended to be used to attach the metaglene 42 in the glenoid.

As described above, FIG. 10 shows a proximal portion of a humerus 16 having a resected metaphyseal surface 10 and a humeral joint part 4′ according to the second variant of the first embodiment. As shown on the figure, the entire anchor component 2 is arranged inside the humerus 16, the proximal face 14 of the proximal flange 12 extends underneath a plane in which the resected metaphyseal surface 10 extends.

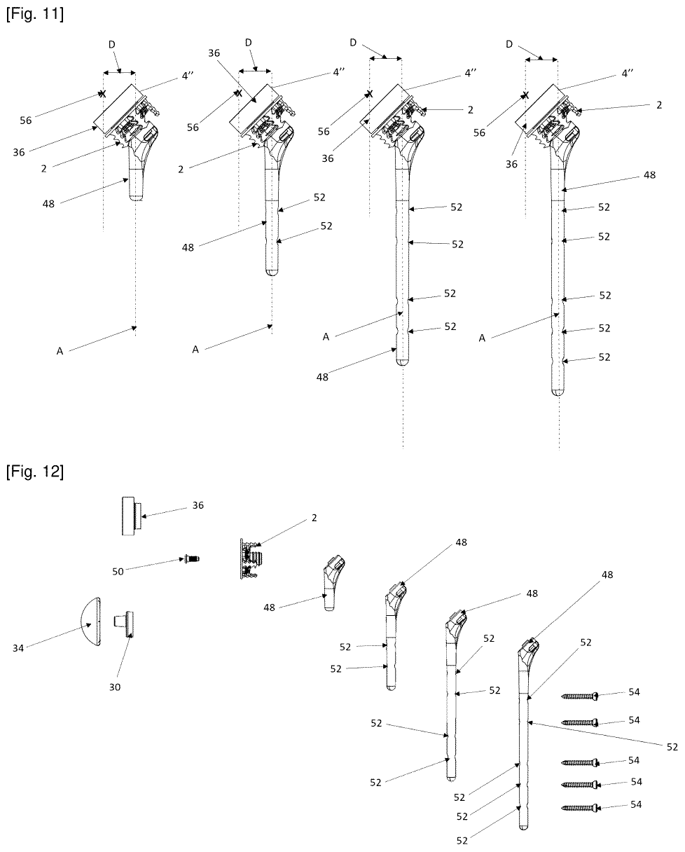

FIGS. 11 and 12 show a second embodiment of a humeral joint part 4″. In the latter, the humeral joint part comprises a humeral anchor component 2 cooperating with a cup 36 to form a humeral joint part 4′. Obviously, a replacement humeral head 34 with a spacer 30 can be placed on the humeral anchor component 2 in order to form a humeral joint part 4.

The humeral joint part 4 or 4′ can be attached to a diaphyseal section, for example a humeral rod 48, for example by means of an attachment screw 50. The length of the diaphyseal section can be different, as shown on FIGS. 11 and 12, with humeral rods of different lengths and depending on the patient who is to receive a humeral joint part 4″ according to the second embodiment.

The use of a diaphyseal section is particularly recommended for a patient whose metaphyseal section of the humerus is damaged. In this situation, attaching a humeral joint part 4 or 4′ according to the first embodiment is hardly feasible since this could make the humeral joint part 4 or 4′ unstable. For stabilization purposes, the diaphyseal section is implanted in the bone, for example by extending a humeral rod 48 in the medullary section of the humerus. A male Morse cone is then placed on the diaphyseal section. The humeral anchor component 2 is then mounted on the diaphyseal section (by interaction between a male Morse cone (mounted on the diaphyseal section) and a female Morse cone (carried by the humeral anchor component 2)) and attached to the latter by means of the attachment screw 50. Once the humeral anchor component 2 is positioned and attached, a cup 36 or a spacer 30/replacement humeral head 34 assembly can be attached to the humeral anchor component 2 to form the humeral joint part 4″. Note that the diaphyseal section could comprise a male cone integrated in the diaphyseal section capable of interacting with the female Morse code carried by the humeral anchor component 2.

Depending on its length, the diaphyseal section, for example the humeral rod 48, can be provided with locking holes 52 cooperating with locking screws 54 to lock the diaphyseal section, and therefore the humeral joint part 4″, in the humerus.

As shown on FIG. 11, the length of the diaphyseal section can be different, with or without locking screws 54, depending on the bone of the patient in which the diaphyseal section is to be implanted. The locking screws 54 are used to stabilize the diaphyseal section when it reaches a certain length.

The disclosed embodiment also relates to a kit for implanting a humeral joint part 4″ according to an embodiment, the kit comprising several humeral anchor components 2 of different diameters and several diaphyseal sections, for example several humeral rods 48, of different lengths. Obviously, the implantation kit can also comprise several cups 36 of different sizes, several replacement humeral heads 34 and several spacers 30 of different sizes and/or several attachment screws 50 of different sizes.

Each humeral anchor component 2 of the implantation kit can be mounted and attached on each diaphyseal section of the implantation kit. The choice of associating a particular humeral anchor component 2 with a particular diaphyseal section depends on the patient, like the choice of using a replacement humeral head 34 or a cup 36 depending on the type of shoulder prosthesis (anatomical or reverse). Depending on the diameter of the humeral anchor component 2, the depth of implantation of the diaphyseal section can be modified so that a humeral anchor component 2 is placed at the same height regardless of the diaphyseal section used. In fact, the greater the diameter of the humeral anchor component 2, the longer the stabilization stud 22. The diaphyseal section must therefore be implanted deeper than with a humeral anchor component 2 of smaller diameter, and therefore with a shorter stabilization stud 22, so that the humeral anchor component 2 is placed at the same height at the metaphyseal section of the humerus.

Concerning the method used to implant a humeral joint part, there are two possibilities:
To implant a humeral joint part 4 or 4′ according to the first embodiment, the humerus is first prepared for the implantation: the humeral head is resected and the metaphyseal section is prepared to receive the humeral anchor component 2. The latter is impacted in the metaphyseal section of the humerus and is stable therein due to the peripheral anchor plates 18 extending in the metaphyseal section of the humerus (additional stability is obtained during the healing by additional components described above, for example the through-holes 26). A spacer 30/replacement humeral head 34 assembly or a cup 36 is then mounted on the humeral anchor component 2 to obtain the humeral joint part 4 or 4′ cooperating respectively with a glenoid implant in the form of a cup (for a humeral joint part 4) or with a glenoid implant comprising a glenosphere (for a humeral joint part 4′). As explained above, the main advantage of the first embodiment lies in the possibility of installing a prosthesis as quickly as possible and in a way that is as least invasive as possible.
To implant a humeral joint part 4″ according to the second embodiment, the humerus is also prepared for the implantation: the humeral head is resected and the metaphyseal section is prepared to receive the humeral anchor component 2. In addition, the diaphyseal section of the humerus is prepared by creating a humeral cavity intended to receive the diaphyseal section of the humeral joint part 4″. To do this, the surgeon chooses a humeral anchor component 2/diaphyseal section assembly adapted to the patient. The surgeon also chooses the male Morse cone to be attached to the diaphyseal section and allowing an interaction with the female Morse cone carried by the humeral anchor component 2. Depending on the diameter of the humeral anchor component 2 and therefore on the length of the stabilization stud 22, the depth of implantation of the diaphyseal section varies, which affects the size and in particular the depth of the humeral cavity created, in order to obtain a humeral anchor component 2 always placed at the same height at the metaphyseal section of the humerus, regardless of its diameter. In particular, the greater the diameter of the humeral anchor component 2, the deeper the diaphyseal section is implanted in the humerus. The aim in this case is to keep an “offset”, corresponding to the distance D between an axis A representing the direction of elongation of the diaphyseal section and the center of rotation 56 of the joint, unchanged regardless of the size of the humeral anchor component 2, and therefore of the stabilization stud 22. Once the diaphyseal section has been installed in the humerus, possibly with the insertion of locking screws 54, the male Morse cone then the humeral anchor component 2 (carrying the female Morse cone) are attached to the diaphyseal section and secured through the use of an attachment screw 50. Lastly, a spacer 30/replacement humeral head 34 assembly or a cup 36 is then mounted on the humeral anchor component 2 to obtain the humeral joint part 4 or 4′ cooperating respectively with a glenoid implant in the form of a cup (for a humeral joint part 4) or with a glenoid implant comprising a glenosphere (for a humeral joint part 4′).

The advantage of having the two embodiments as disclosed herein is also to allow a surgeon to adapt to each case. It is not possible, in fact, to determine the bone quality of a patient in advance. Consequently, having a kit comprising at the same time the humeral anchor component 2 that can be implanted alone but also with a diaphyseal section allows the surgeon to adapt with a single product to all situations. If the patient's bone is healthy or in case of deformation of the humerus and preventing the installation of a diaphyseal section, only a humeral anchor component 2 is installed. If on the contrary the patient's bone is of poor quality but in which a diaphyseal section can be implanted, then a diaphyseal section/humeral anchor component 2 assembly can be installed. For example, if a surgeon intends to install a humeral anchor component 2 alone but finds that this is not possible considering the patient's bone, they can adapt by adding a diaphyseal section (and a male Morse cone as well as an attachment screw 50) to switch to the second embodiment, keeping the same kit (i.e. without having to switch to another prosthesis, for example of different design).

What is claimed is:

1. A humeral anchor component of a humeral joint part of a shoulder prosthesis, wherein the humeral anchor component comprises:
a main body adapted to be inserted inside a humerus from a resected metaphyseal surface, and
a proximal flange forming a proximal end of the humeral anchor component and placed at one end of the main body, a proximal face of the proximal flange extending in the humerus and underneath the resected metaphyseal surface.

2. The humeral anchor component according to claim 1, wherein the main body is adapted to extend only in a metaphyseal section of the humerus, the main body comprising a peripheral anchor system formed by several peripheral anchor plates separated from each other and adapted to extend from the proximal flange towards a distal section of the humerus, two adjacent peripheral anchor plates extending in planes that are not parallel to each other.

3. The humeral anchor component according to claim 2, wherein a width of the peripheral anchor plates is irregular along their extension direction.

4. The humeral anchor component according to claim 1, wherein the main body comprises at least one stabilization stud adapted to extend from the proximal flange towards a distal section of the humerus.

5. The humeral anchor component according to claim 4, wherein the at least one stabilization stud comprises retention grooves extending substantially parallel to a longitudinal axis of the at least one stabilization stud.

6. The humeral anchor component according to claim 4, wherein the stabilization stud is a hollow tubular body open at its proximal end and comprises a tapping formed on an internal surface of the stabilization stud.

7. The humeral anchor component according to claim 4, wherein the stabilization stud comprises at least one lateral ring extending from an external face of the stabilization stud in a direction substantially perpendicular to a longitudinal axis of the stabilization stud.

8. A humeral joint part comprising the humeral anchor component according to claim 1.

9. The humeral joint part according to claim 8, further comprising: a diaphyseal section on which the humeral anchor component is attached.

10. The humeral joint part according to claim 9, wherein the diaphyseal section is formed by a humeral rod.

11. A shoulder prosthesis comprising: a humeral joint part, wherein the humeral joint part comprises the humeral anchor component according to claim 1.

12. An implantation kit for implanting a humeral joint part according claim 1, comprising: several humeral anchor components, wherein the several humeral anchor components are the humeral anchor component according to claim 1 of different diameters; and several diaphyseal sections of different lengths.

13. The implantation kit according to claim 12, further comprising: at least one attachment screw for attaching a diaphyseal section to a humeral anchor component.

14. The implantation kit according to claim 12, further comprising: at least one spacer, at least one replacement humeral head and/or at least one cup.

15. The implantation kit according to claim claim 12, comprising: locking screws for locking the diaphyseal section in a humerus in which the diaphyseal section is implanted.

16. A method of implanting a humeral joint part, comprising:
resecting a humeral head of a humerus;
resecting a metaphyseal surface at a metaphyseal section of the humerus;
impacting a humeral anchor component 2 in the resected metaphyseal surface of the humerus, wherein a peripheral anchor plate 18 is adapted to extend in the metaphyseal section of the humerus; and
mounting an assembly of a spacer 30 and a replacement humeral head 34 on the humeral anchor component 2 to obtain the humeral joint part 4 or 4′, wherein the humeral joint part 4 or 4′ cooperates respectively with a glenoid implant in a form of a cup,
wherein the humeral anchor component 2 comprises:
a main body adapted to be inserted inside the humerus from the resected metaphyseal surface, and
a proximal flange forming a proximal end of the humeral anchor component and placed at one end of the main body, a proximal face of the proximal flange extending in the humerus and underneath the resected metaphyseal surface.

17. The method of claim 16, further comprising:
creating a humeral cavity on a diaphyseal section of the humerus, wherein the humeral cavity is adapted to receive a diaphyseal section of the humeral joint part 4″, wherein a depth of the humeral cavity is determined based on a diameter of the humeral anchor component 2 and a length of a stabilization stud 22, wherein the humeral anchor component is adapted to be placed at a same height at the metaphyseal section of the humerus;
selecting a humeral anchor component 2 and the diaphyseal section of the humeral joint part, wherein a length of the diaphyseal section of the humeral joint part is adapted to a patient;
installing the diaphyseal section of the humeral joint part in the humeral cavity of the humerus;
attaching a male morse cone to the diaphyseal section of the humeral joint part, wherein the male morse cone is adapted to interact with a female morse cone of the humeral anchor component 2;
attaching the humeral anchor component 2 with the female morse cone to the diaphyseal section of the humeral joint via the male morse; and
securing the attached humeral anchor component 2 and the diaphyseal section with an attachment screw 50.

18. The method of claim 16, where in the peripheral anchor plate and through-holes of the humeral anchor component is adapted to provide stability during healing.

19. The method of claim 16, wherein a cap 36 is used in place of the assembly of the spacer and the replacement humeral head, thereby mounting the cap on the humeral anchor component 2.

20. The method of claim 17, wherein installing the diaphyseal section of the humeral joint part in the humeral cavity of the humerus further comprises inserting locking screws to locking holes for stability.
